(19) United States
(12) Patent Application Publication
(10) Pub. No.: US 2024/0115510 A1
(43) Pub. Date: Apr. 11, 2024

(54) TESTIS-TARGETED LYCOPENE (LYC)/ZIF-90 NANOCOMPOSITE AND PREPARATION METHOD AND USE THEREOF

(71) Applicant: NORTHEAST AGRICULTURAL UNIVERSITY, Heilongjiang (CN)

(72) Inventors: Jinlong LI, Heilongjiang (CN); Haoran WANG, Heilongjiang (CN); Muzi LI, Heilongjiang (CN); Yi ZHAO, Heilongjiang (CN); Xuenan LI, Heilongjiang (CN); Lin LIU, Heilongjiang (CN); Shiyong ZHU, Heilongjiang (CN)

(21) Appl. No.: 18/084,069

(22) Filed: Dec. 19, 2022

(30) Foreign Application Priority Data
Oct. 9, 2022 (CN) 202211224505.7

(51) Int. Cl. A61K 9/51; A61K 31/01; A61K 38/24; A61P 15/08

(52) U.S. Cl. CPC A61K 9/5115; A61K 9/5123; A61K 9/5192; A61K 31/01; A61K 38/24; A61P 15/08

(57) ABSTRACT

The present disclosure provides a testis-targeted lycopene (LYC)/ZIF-90 nanocomposite and a preparation method and use thereof, belonging to the technical field of targeted drugs. In the present disclosure, a ZIF-90 metal-organic framework (MOF) material is combined with LYC through aldol condensation; when entering areas of inflammatory response and oxidative stress response, ZIF-90 may disintegrate and collapse in a slightly-acidic environment, releasing the LYC, thereby avoiding easy oxidation of the LYC and improving a bioavailability of the LYC; in addition, the disintegrated and collapsed ZIF-90 may also be utilized by organisms, thus greatly reducing a damage to the organisms. Moreover, the LYC/ZIF-90 nanocomposite is further loaded with follicle-stimulating hormone (FSH). The FSH can specifically bind to a follicle-stimulating hormone receptor (FSHR) of a Sertoli cell to improve testicular targeting of the LYC/ZIF-90 nanocomposite, thereby improving a therapeutic effect of male reproductive dysfunction.

### CROSS-REFERENCE TO RELATED APPLICATIONS

This patent application claims the benefit of and priority to Chinese Patent Application No. 202211224505.7, filed with the China National Intellectual Property Administration on Oct. 9, 2022, the disclosure of which is incorporated by reference herein in its entirety as part of the present application.

### TECHNICAL FIELD

The present disclosure relates to the technical field of targeted drugs, in particular to a testis-targeted lycopene (LYC)/ZIF-90 nanocomposite and a preparation method and use thereof.

### BACKGROUND

Male reproductive health has been recognized as a global health problem. Factors such as increasing age, obesity, poor diet, and environmental toxicants can contribute to the male reproductive health problems. In aerobic organisms, there are a lot of oxidative and antioxidant substances. Reactive oxygen species (ROS) with an appropriate concentration may act as signaling molecules to participate in the maintenance of many cellular life activities, but excessive ROS can lead to the imbalance of oxidation and antioxidation in the body. Endogenous ROS is a main source of ROS in vivo, mainly produced in mammalian mitochondria. In addition to the endogenous pathway, the male reproductive system can also be exposed to various exogenous stimuli (such as exposure to environmental pollutants including chemical reagents, PM2.5, and heavy metals, as well as bad habits including smoking and drinking) to generate ROS, in turn causing a series of oxidative stress damages.

Lycopene (LYC), with a molecular formula C₄₀H₅₆, is a known carotenoid with the strongest antioxidant capacity in nature. LYC is found in red fruits and vegetables such as tomatoes, watermelons, pink grapefruits, apricots, papayas, cranberries, guava, and peaches. The LYC has 11 conjugated double bonds with a strong ROS quenching ability, and can protect cells from the oxidation of free radical components by removing the free radicals. LYC is far more effective at scavenging free radicals than other carotenoids and vitamin E, and has a rate constant for quenching singlet oxygen 100 times that of vitamin E. Several studies suggest that LYC may play a major physiological role as an antioxidant during spermatogenesis and may be helpful in the treatment of male infertility. LYC also reduces sperm DNA fragmentation and inhibits lipid peroxidation in infertile men through its antioxidant activity. However, the LYC has a poor chemical stability and is easily oxidized, leading to low bioavailability.

### SUMMARY

In view of this, an objective of the present disclosure is to provide a testis-targeted LYC/ZIF-90 nanocomposite and a preparation method and use thereof. The testis-targeted LYC/ZIF-90 nanocomposite has a desirable bioavailability and a therapeutic effect on male reproductive dysfunction.

To achieve the above objective of the present disclosure, the present disclosure provides the following technical solutions.

The present disclosure provides a testis-targeted LYC/ZIF-90 nanocomposite, including an LYC/ZIF-90 nanocomposite and follicle-stimulating hormone (FSH) loaded on surface and internal pores of the LYC-ZIF-90 nanocomposite; where
the LYC/ZIF-90 nanocomposite includes a ZIF-90 metal-organic framework (MOF) material and LYC bound to the ZIF-90 MOF material through aldol condensation.

Preferably, the ZIF-90 MOF material and the LYC have a mass ratio of 100:(10-15).

Preferably, the ZIF-90 MOF material and the FSH have a mass ratio of 100:(5-10).

Preferably, the ZIF-90 MOF material has a pore size of 1 nm to 3 nm.

Preferably, the testis-targeted LYC/ZIF-90 nanocomposite has a particle size of 100 nm to 150 nm.

The present disclosure further provides a preparation method of the testis-targeted LYC/ZIF-90 nanocomposite, including the following steps:
mixing the ZIF-90 MOF material and the LYC with an alcohol solvent to conduct aldol condensation to obtain an LYC/ZIF-90 nanocomposite; and
mixing the LYC/ZIF-90 nanocomposite and the FSH with the alcohol solvent to conduct loading to obtain the testis-targeted LYC/ZIF-90 nanocomposite.

Preferably, the aldol condensation is conducted at 25° C. to 30° C. for 36 h to 48 h.

Preferably, a preparation method of the ZIF-90 MOF material includes the following steps:
mixing an imidazole-2-carboxaldehyde solution, polyvinylpyrrolidone (PVP), and a zinc source solution to conduct complexation to obtain the ZIF-90 MOF material.

Preferably, the loading is conducted at 25° C. to 30° C. for 12 h to 24 h.

The present disclosure further provides use of the testis-targeted LYC/ZIF-90 nanocomposite in preparation of a drug for treating male reproductive dysfunction.

The present disclosure provides a testis-targeted LYC/ZIF-90 nanocomposite, including an LYC/ZIF-90 nanocomposite and FSH loaded on a surface and internal pores of the LYC-ZIF-90 nanocomposite; where the LYC/ZIF-90 nanocomposite includes a ZIF-90 MOF material and LYC bound to the ZIF-90 MOF material through aldol condensation. In the present disclosure, the ZIF-90 MOF material is used as a substrate for loading the LYC, has a desirable biocompatibility, and does not affect the normal in vivo environment as a drug carrier after entering the human body; meanwhile, ZIF-90 can also improve a biological stability of the LYC. This is because the ZIF-90 contains Zn²⁺ for complexation, and since the complexation between Zn²⁺ and cellular adenosine triphosphate (ATP) is stronger than that between imidazole and Zn²⁺, the ZIF-90 can decompose in response to the ATP. In addition, the ZIF-90 MOF material has pH-responsive properties, which can be dissociated in a slightly-acidic environment (with a pH value of 5 to 6), but still maintain a structural stability under normal physiological conditions. When the testicular tissue produces inflammation, oxidative stress and apoptosis, leading to abnormal cell microenvironment, mitochondria may produce a large amount of ROS and ATP, resulting in a slightly-acidic cell environment. The ZIF-90 MOF material is combined with the LYC through aldol condensation; when entering areas of inflammatory response and oxidative stress response, the ZIF-90 MOF material may disintegrate and collapse in the slightly-acidic environment, releasing the LYC, thereby avoiding easy oxidation of the LYC and improving a bioavailability of the LYC; in addition, the disintegrated and collapsed ZIF-90 may also be decomposed to release Zn²⁺ that can be used as a therapeutic agent to overcome tumor resistance to anticancer drugs, and to modulate a series of physiological responses to inhibit tumor growth. Furthermore, Zn²⁺, as an essential trace element in the body, can also maintain the normal appetite of the human body, enhance the immunity of the body, and contribute to the healing of the wounds and traumata. Moreover, the LYC/ZIF-90 nanocomposite is further loaded with FSH. The FSH can specifically bind to a follicle-stimulating hormone receptor (FSHR) of a Sertoli cell to improve testicular targeting of the LYC/ZIF-90 nanocomposite, thereby improving a therapeutic effect of male reproductive dysfunction.

### BRIEF DESCRIPTION OF THE DRAWINGS

FIG. 1 shows transmission electron microscopy (TEM) images of ZIF-90 and LYC @ZIF-90-FSH in Example 3;

FIG. 2 shows a particle size analysis, particle dispersion index (PDI) and Zeta potential of the ZIF-90 and the LYC @ZIF-90-FSH in Example 3;

FIG. 3 shows an X-ray diffraction (XRD) spectrum of the ZIF-90 and the LYC @ZIF-90-FSH in Example 3;

FIG. 4 shows Fourier-transform infrared spectroscopy (FTIR) spectrum of the ZIF-90 and the LYC @ZIF-90-FSH in Example 3;

FIG. 5 shows a result analysis on the toxicity of an LYC@ZIF-90-FSH nano-drug delivery system to various cells;

FIG. 6 shows a distribution of the LYC @ZIF-90-FSH in TM4 cells;

FIG. 7 shows a drug release rate analysis of the LYC @ZIF-90-FSH in ATP and acidic pH environments in vitro;

FIG. 8 shows a test result of the LYC @ZIF-90-FSH targeting mouse testis tissues;

FIG. 9 shows a content of anti-inflammatory factors in sera and tissues under different materials; and

FIG. 10 shows a result of HE staining.

### DETAILED DESCRIPTION

The present disclosure provides a testis-targeted LYC/ZIF-90 nanocomposite, including an LYC/ZIF-90 nanocomposite and follicle-stimulating hormone (FSH) loaded on a surface and internal pores of the LYC-ZIF-90 nanocomposite; where
the LYC/ZIF-90 nanocomposite includes a ZIF-90 metal-organic framework (MOF) material and LYC bound to the ZIF-90 MOF material through aldol condensation.

In the present disclosure, a preparation method of the ZIF-90 MOF material includes preferably the following steps:
mixing an imidazole-2-carboxaldehyde solution, PVP, and a zinc source solution to conduct complexation to obtain the ZIF-90 MOF material.

In the present disclosure, a solvent of the imidazole-2-carboxaldehyde solution is preferably a mixed solution of glycerol and water; and in the mixed solution of glycerol and water, the glycerol has a mass content of preferably 50%. In the imidazole-2-carboxaldehyde solution, a mass of the imidazole-2-carboxaldehyde and a volume of the solvent have a ratio of preferably 0.48 g:(10-20) mL, more preferably 0.48 g:15 mL. Preferably, the imidazole-2-carboxaldehyde is dissolved in the solvent by conducting an ultrasonic treatment for preferably 30 min to 50 min, more preferably 40 min.

In the present disclosure, the zinc source is preferably zinc nitrate, more preferably zinc nitrate hexahydrate. A solvent of the zinc source solution is preferably tert-butanol. In the zinc source solution, a mass of the zinc source and a volume of the solvent have a ratio of preferably (0.3-0.4) g:(10-20) mL, more preferably 0.48 g:15 mL. Preferably, the zinc source is dissolved in the solvent by conducting an ultrasonic treatment for preferably 30 min to 50 min, more preferably 40 min.

In the present disclosure, the imidazole-2-carboxaldehyde, the PVP, and the zinc source have a mass ratio of preferably 0.48:(0.05-0.1):(0.3-0.4).

In the present disclosure, as a surfactant, the PVP has better colloidal dispersibility and stability; and after adding the PVP, nanoparticles may have s smaller particle size and better dispersion.

In the present disclosure, the complexation is conducted at preferably a room temperature for preferably 15 min to 30 min, more preferably 20 min to 25 min. The complexation is conducted preferably under stirring at preferably 700 rpm to 1,000 rpm, more preferably 800 rpm to 900 rpm.

In the present disclosure, the ZIF-90 MOF material has a pore size of preferably 1 nm to 3 nm, more preferably 2 nm to 3 nm.

In the present disclosure, the ZIF-90 MOF material and the LYC have a mass ratio of preferably 100:(10-15), more preferably 100:(12-14).

In the present disclosure, the ZIF-90 MOF material and the FSH have a mass ratio of preferably 100:(5-10), more preferably 100:(6-8).

In the present disclosure, the testis-targeted LYC/ZIF-90 nanocomposite has a particle size of preferably 100 nm to 150 nm, more preferably 120 nm to 140 nm.

The present disclosure further provides a preparation method of the testis-targeted LYC/ZIF-90 nanocomposite, including the following steps:
mixing the ZIF-90 MOF material and the LYC with an alcohol solvent to conduct aldol condensation to obtain an LYC/ZIF-90 nanocomposite; and
mixing the LYC/ZIF-90 nanocomposite and the FSH with the alcohol solvent to conduct loading to obtain the testis-targeted LYC/ZIF-90 nanocomposite.

In the present disclosure, the ZIF-90 MOF material and the LYC are mixed with an alcohol solvent to conduct aldol condensation to obtain an LYC/ZIF-90 nanocomposite, denoted as LYC @ZIF-90. A preparation method of the ZIF-90 MOF material is the same as the above, and is not repeated here.

In the present disclosure, the alcohol solvent I is preferably ethanol, more preferably absolute ethanol.

In the present disclosure, the ZIF-90 MOF material and the LYC have a mass ratio of preferably 100:(10-15), more preferably 100:(12-14).

In the present disclosure, the mixing is conducted by preferably an ultrasonic treatment at preferably 100 W for preferably 30 min to 50 min, more preferably 40 min.

In the present disclosure, the aldol condensation is conducted preferably in the dark at preferably 25° C. to 30° C. for preferably 36 h to 48 h, more preferably 40 h to 45 h.

In the present disclosure, the aldol condensation is conducted preferably under stirring at preferably 100 rpm to 200 rpm, more preferably 150 rpm.

In the present disclosure, during the aldol condensation, an aldehyde group of the ZIF-90 and a hydroxyl group of the LYC are ligated through condensation.

In the present disclosure, after the aldol condensation, preferably an obtained aldol condensation reaction solution is subjected to a post-treatment, preferably including the following steps:
subjecting the aldol condensation reaction solution to solid-liquid separation, and washing and drying an obtained solid to obtain an LYC/ZIF-90 nanocomposite solid.

In the present disclosure, the solid-liquid separation is conducted by preferably centrifugation. The washing is conducted preferably 3 times with preferably ethanol as a detergent; and the drying is conducted by preferably freeze-drying.

In the present disclosure, the LYC/ZIF-90 nanocomposite and the FSH are mixed with the alcohol solvent to conduct loading to obtain the testis-targeted LYC/ZIF-90 nanocomposite, denoted as LYC@ZIF-90-FSH.

In the present disclosure, the alcohol solvent II is preferably methanol, more preferably anhydrous methanol.

In the present disclosure, the ZIF-90 MOF material and the FSH have a mass ratio of preferably 100:(5-10), more preferably 100:(6-8).

In the present disclosure, the mixing is conducted by preferably an ultrasonic treatment at preferably 100 W for preferably 30 min to 50 min, more preferably 40 min.

In the present disclosure, the loading is conducted preferably in the dark under nitrogen protection at preferably 25° C. to 30° C. for preferably 12 h to 24 h, more preferably 16 h to 20 h. In the present disclosure, the loading is conducted preferably under stirring at preferably 100 rpm to 200 rpm, more preferably 150 rpm.

In the present disclosure, during the loading, the FSH is adsorbed on a surface and internal pores of the LYC/ZIF-90 nanocomposite.

In the present disclosure, after the loading, preferably an obtained loading solution is subjected to a post-treatment, preferably including the following steps:
subjecting the loading solution to solid-liquid separation, and washing and drying an obtained solid in sequence to obtain a testis-targeted LYC/ZIF-90 nanocomposite solid. In the present disclosure, the solid-liquid separation is conducted by preferably centrifugation; the washing is conducted preferably 3 times with preferably methanol as a detergent; and the drying is conducted by preferably freeze-drying for preferably 12 h.

The present disclosure further provides use of the testis-targeted LYC/ZIF-90 nanocomposite in preparation of a drug for treating male reproductive dysfunction. The testis-targeted LYC/ZIF-90 nanocomposite can alleviate oxidative stress and inflammatory damages caused by various reproductive system diseases.

In the present disclosure, the drug for treating male reproductive dysfunction is preferably a drug for treating testicular cancer and testicular infection.

In the present disclosure, the testis-targeted LYC/ZIF-90 nanocomposite is preferably labeled with fluorescein when preparing the drug for treating male reproductive dysfunction. The fluorescein is preferably Rhodamine B.

The testis-targeted LYC/ZIF-90 nanocomposite and the preparation method and the use thereof as provided by the present disclosure will be described in detail in connection with the following examples, but they should not be construed as limiting the claimed scope of the present disclosure.

### Example 1

A preparation method of LYC@ZIF-90-FSH included the following steps:

(1) Preparation of a ZIF-90 MOF Material:
371.25 mg of zinc nitrate and 8 mg of tert-butanol were added to 10 mL of an aqueous solution to obtain a solution A;
480 mg of imidazole-2-carboxaldehyde (2-ICA) and 50 mg of PVP were added to 10 mL of the aqueous solution to obtain a solution B; and
after conducting an ultrasonic treatment, the solution A was slowly added to the solution B, and a resulting mixed solution was stirred under inert gas protection at a room temperature for 10 min to obtain a pale yellow precipitate. The mixed solution was centrifuged three times at 14,000 g for 30 min to obtain a precipitate, and the precipitate was washed with excess methanol and dried under vacuum at −55° C. for 24 h.

(2) Preparation of LYC @ZIF-90:
50 mg of LYC was dissolved in 10 mL of methanol in advance to obtain a methanol solution with a concentration of 5 mg/mL, 0.1 g of the ZIF-90 MOF material prepared in step (1) was added dropwise and dissolved in 10 mL of the methanol solution, and a resulting mixture was reacted by stirring under inert gas protection at a room temperature and 200 rpm/min for 48 h. An obtained solution was centrifuged three times at 14,000 g for 30 min to obtain a precipitate, and the precipitate was washed with excess methanol, and then freeze-dried to obtain a powder, namely the LYC @ZIF-90.

(3) Preparation of LYC@ZIF-90-FSH:
0.1 g of the LYC @ZIF-90 prepared in step (2) was redissolved in a PBS. 10 mg of FSH was dissolved in 10 mL of methanol, added to a PBS with LYC @ZIF-90, and then sonicated at a room temperature for 30 min until completely dissolved; an obtained product was washed and centrifuged with excess PBS, put into a dialysis bag to exchange with the PBS for 48 h in the dark, and then freeze-dried for 12 h to obtain the LYC@ZIF-90-FSH.

### Example 2

(1) Preparation of a ZIF-90 MOF Material:
371.25 mg of zinc nitrate and 4 mg of tert-butanol were added to 10 mL of an aqueous solution to obtain a solution A;
480 mg of imidazole-2-carboxaldehyde (2-ICA) and 50 mg of PVP were added to 10 mL of the aqueous solution to obtain a solution B; and
after conducting an ultrasonic treatment, the solution A was slowly added to the solution B, and a resulting mixed solution was stirred under inert gas protection at a room temperature for 10 min to obtain a pale yellow precipitate. The mixed solution was centrifuged three times at 14,000 g for 30 min to obtain a precipitate, and the precipitate was washed with excess methanol and dried under vacuum at −55° C. for 24 h.

(2) Preparation of LYC @ZIF-90:
15 mg of LYC was dissolved in 3 mL of methanol in advance to obtain a methanol solution with a concentration of 5 mg/mL, 0.1 g of the ZIF-90 MOF material prepared in step (1) was added dropwise and dissolved in 10 mL of the methanol solution, and a resulting mixture was reacted by stirring under inert gas protection at a room temperature and 200 rpm/min for 48 h. An obtained solution was centrifuged three times at 14,000 g for 30 min to obtain a precipitate, and the precipitate was washed with excess methanol, and then freeze-dried to obtain a powder, namely the LYC @ZIF-90.

(3) Preparation of LYC@ZIF-90-FSH:
0.1 g of the LYC @ZIF-90 prepared in step (2) was redissolved in methanol. 10 mg of FSH was dissolved in 10 mL of methanol, added to a PBS with LYC @ZIF-90, and then sonicated at a room temperature for 30 min until completely dissolved; an obtained product was washed and centrifuged with excess PBS, put into a dialysis bag to exchange with the PBS for 48 h in the dark, and then freeze-dried for 12 h to obtain the LYC@ZIF-90-FSH.

### Example 3

(1) Preparation of a ZIF-90 MOF Material:
371.25 mg of zinc nitrate was added to 10 mL of a tert-butanol solution to obtain a solution A;
480 mg of 2-ICA and 50 mg of PVP were added to 10 mL of a methanol solution to obtain a solution B; and
after conducting an ultrasonic treatment, the solution A was slowly added to the solution B, and a resulting mixed solution was stirred under nitrogen protection at a room temperature for 10 min to obtain a pale yellow precipitate. The mixed solution was centrifuged three times at 12,000 rpm for 30 min to obtain a precipitate, and the precipitate was washed with excess methanol and dried under vacuum at −55° C. for 24 h.

(2) Preparation of LYC @ ZIF-90:
15 mg of LYC was dissolved in 3 mL of methanol in advance to obtain a methanol solution with a concentration of 5 mg/mL, 0.1 g of the ZIF-90 MOF material prepared in step (1) was added dropwise and dissolved in 10 mL of the methanol solution, and a resulting mixture was reacted by stirring under inert gas protection at a room temperature and 200 rpm/min for 48 h. An obtained solution was centrifuged three times at 12,000 g for 30 min to obtain a precipitate, and the precipitate was washed with excess methanol, and then freeze-dried to obtain a powder, namely the LYC @ZIF-90.

(3) Preparation of LYC@ZIF-90-FSH:
0.1 g of the LYC @ZIF-90 prepared in step (2) was redissolved in methanol. 10 mg of FSH was dissolved in 10 mL of methanol, added to a PBS with LYC @ZIF-90, and then sonicated at a room temperature for 30 min until completely dissolved; an obtained product was washed and centrifuged with excess PBS, put into a dialysis bag to exchange with the PBS for 48 h in the dark, and then freeze-dried for 12 h to obtain the LYC@ZIF-90-FSH, abbreviated as LZF.

### Test Example 1

According to the drug-loading system LYC@ZIF-90-FSH prepared in Example 3, a series of performance testing tests were conducted on the drug-loading system LYC@ZIF-90-FSH, including transmission electron microscopy (TEM) detection, particle dispersion index (PDI), Zeta potential, X-ray diffraction (XRD) instrument analysis, and infrared spectroscopy (IR) detection.

(1) TEM Detection

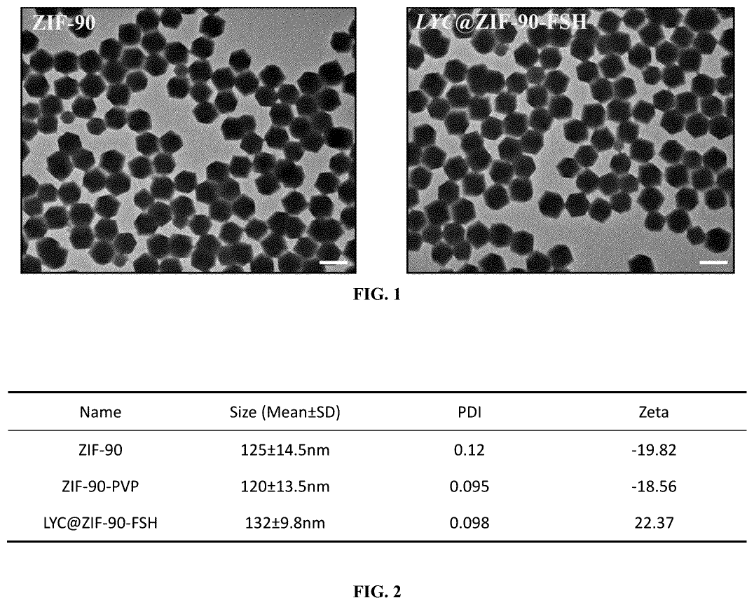

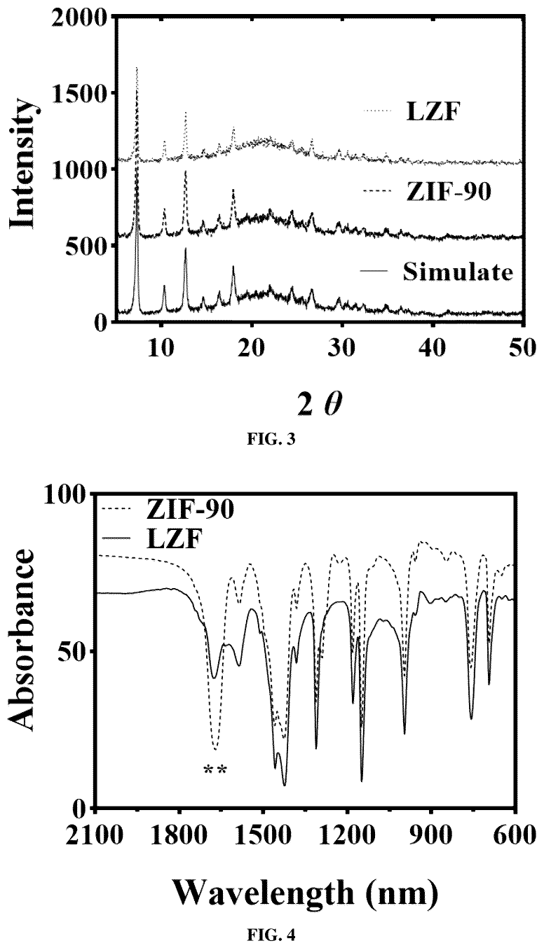

In order to analyze the morphology and size of synthesized ZIF-90 and LYC @ZIF-90-FSH, TEM characterization was conducted. A certain amount of the product powder was weighed separately, dispersed with absolute ethanol, added dropwise to a copper mesh, and dried; a product-loaded copper mesh was observed under a TEM. The results were shown in FIG. 1, and a particle size analysis, PDI and Zeta potential were shown in FIG. 2.

It was seen from FIG. 1 to FIG. 2 that the ZIF-90 and LYC@ZIF-90-FSH each were uniformly distributed in a hexahedron, with a particle size of about 100 nm to 150 nm and desirable uniformity, after loading LYC and FSH, the average particle size of ZIF-90 nanoparticles increased slightly. This further indicated that the LYC and FSH had a little effect on the structure and size of ZIF-90 materials. In addition, the Zeta potential was detected, and it was found that the LYC@ZIF-90-FSH surface of the nanoparticles is positively charged, because the negatively charged ZIF-90 interacts with the positively charged FSH, and the charge of the nanoparticles turns from negative to positive. Finally, the nanoparticles kept relatively uniform average particle size and dispersion within two weeks, which proved that they had good stability.

(2) XRD Instrument Analysis

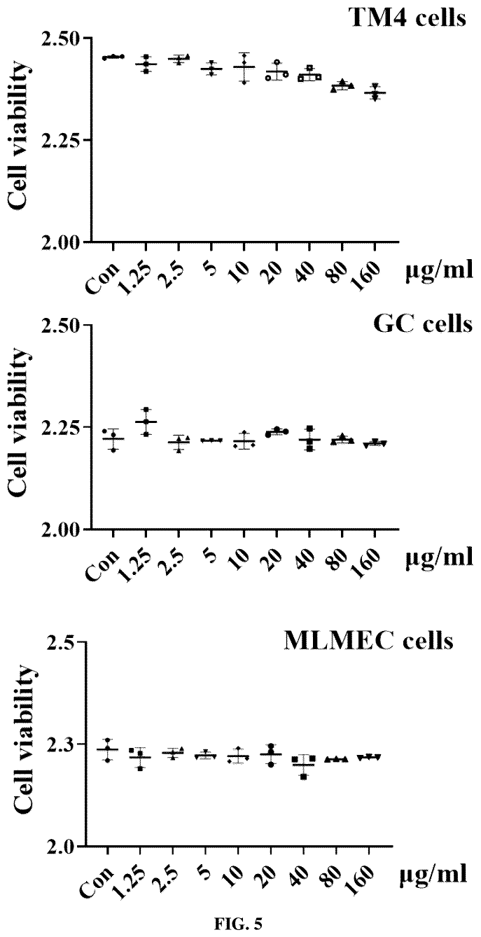

The crystal structures of ZIF-90 and LYC@ZIF-90-FSH were verified by XRD, and the results were shown in FIG. 3. Compared with a simulated XRD peak, LYC@ZIF-90-FSH nanoparticles obtained in the experiment were basically consistent with peak positions of the simulated spectrum, and there were no additional impurity peaks, proving that the LYC @ZIF-90-FSH nano-drug delivery system was successfully prepared with less impurities.

(3) FT-IR Detection

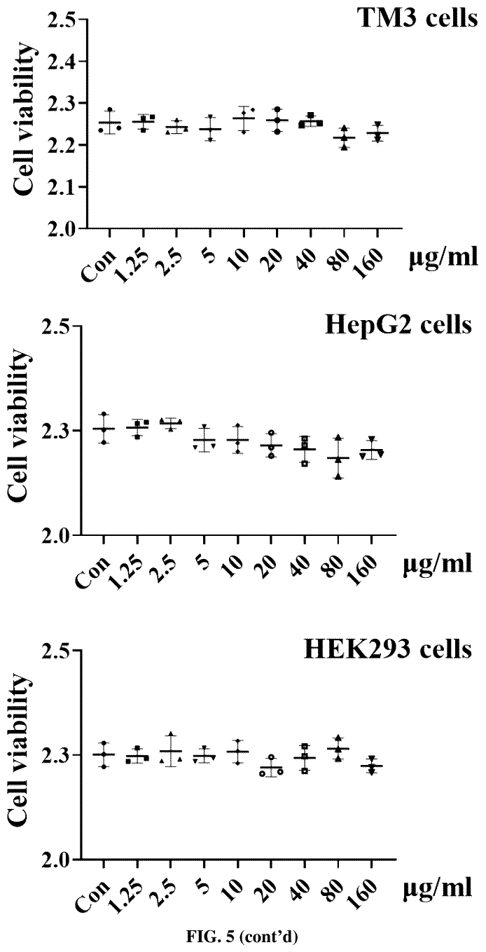

The ZIF-90 and LYC@ZIF-90-FSH were mixed and ground with potassium bromide separately, pressed into tablets, and detected by FT-IR. The results were shown in FIG. 4. FIG. 4 showed FT-IR spectra of the ZIF-90 and LYC@ZIF-90-FSH. According to a position of each characteristic peak, the LYC@ZIF-90-FSH material was successfully synthesized, and a aldehyde group in the material was not destroyed.

### Test Example 2 Study on Toxicity of LYC@ZIF-90-FSH to Various Cells

A preparation method of the LYC@ZIF-90-FSH was the same as that in Example 3.

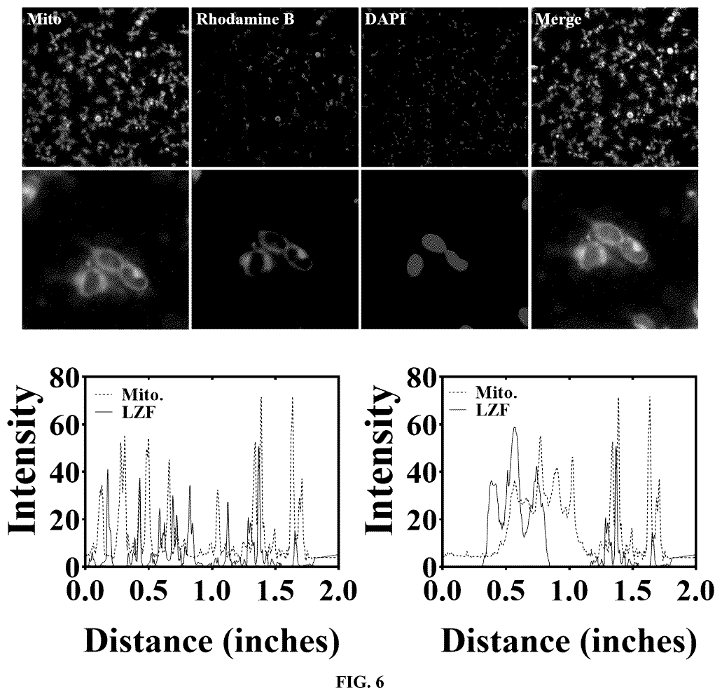

Various cells (GC, TM3, TM4, HEK293, HepG2, and MLMEC) were inoculated into a 96-well culture plate, and after the cells adhered, the cells were treated with corresponding concentrations of RGD/PTX@ZIF-90 (0 μM, 1.25 μM, 2.5 μM, 5 μM, 10 μM, 20 μM, 40 μM, 80 μM, and 160 μM) for 24 h. 100 μL of a CCK8 working solution was added to each well, the cells were incubated for 1 h in a cell incubator, and an absorbance of each well was detected at a wavelength of 450 nm on a microplate reader. The results were shown in FIG. 5. As was seen from FIG. 5, the material itself was not toxic to cells.

### Test Example 3 Observation on Distribution of LYC@ZIF-90-FSH in TM4 Cells

A preparation method of the LYC@ZIF-90-FSH was the same as that in Example 3, except that the nanoparticles were labeled with Rhodamine B.

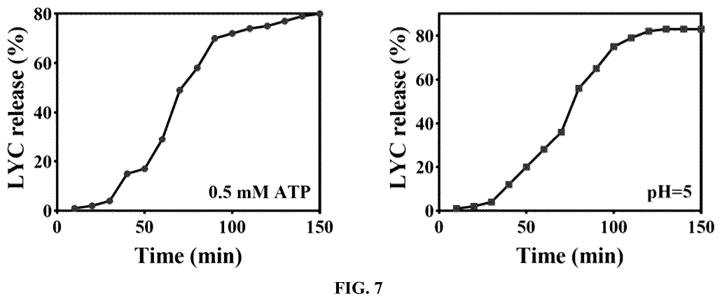

In order to more intuitively prove that the LYC@ZIF-90-FSH could enter TM4 cells smoothly, DAPI-labeled TM4 cells were used as experimental cells for experiments, and intracellular release and distribution of the LYC@ZIF-90-FSH were observed by a fluorescence microscope. The results were shown in FIG. 6. As was seen from FIG. 6, after co-incubating TM4 cells with the LYC@ZIF-90-FSH for 1.5 h, the intracellular fluorescence of LYC@ZIF-90-FSH was successfully captured by the fluorescence microscope. This indicated that the LYC @ZIF-90-FSH drug-loading system could be rapidly taken up by TM4 cells and successfully entered into the cells.

### Test Example 4 ATP- and pH-Responsive Drug Release of LYC@ZIF-90-FSH In Vitro

A preparation method of the LYC @ZIF-90-FSH drug delivery system was the same as that in Example 3.

To test whether the LYC@ZIF-90-FSH could release drug in response to ATP and pH in vitro, a method included the following steps:
2 mg of a dried LYC@ZIF-90-FSH powder was separately poured into two 10 mL centrifuge tubes, one with 2 mL of a pH=5 PBS buffer, and the other with 2 mL of a pH=7 PBS buffer, and two obtained solution were loaded by two dialysis bags after dissolving. The two dialysis bags were placed in two beakers containing 20 mL of buffer solutions with their respective pH value; the beakers were rotated at a low speed on a magnetic stirrer; 1 mL of the PBS buffer was periodically pipetted from each of the two beakers and measured at a wavelength of 502 nm; meanwhile, 1 mL of the PBS buffer at the corresponding pH was added to the two beakers in time, such that a volume of the buffer in each beaker was the same as that before pipetting. An amount of drug released in the two beakers over a period of time was calculated by the measured absorbances. An ATP screening method was the same as above, except that the solutions were a 0.5 mM ATP PBS and a 0 mM ATP PBS.

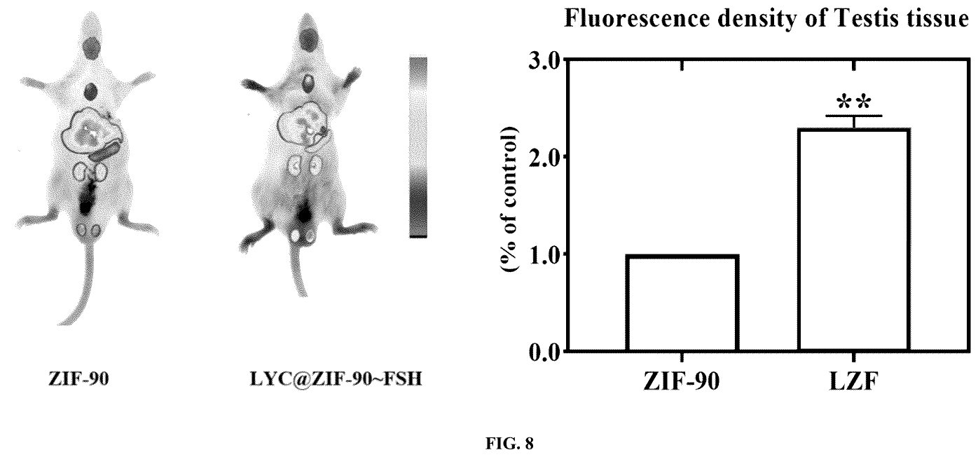

After screening, the drug release rate was higher in both environments of 0.5 mM ATP and pH=5.0 PBS, as shown in FIG. 7. As was seen from FIG. 7, the LYC@ZIF-90-FSH in the presence of acid and ATP promoted the release of LYC in cells with high ROS and ATP levels.

### Test Example 5 Test of LYC@ZIF-90-FSH Targeting Testicular Tissues

A preparation method of the LYC @ZIF-90-FSH drug delivery system was the same as that in Example 3, except that the nanoparticles were labeled with Cy5.5.

In order to further test whether the LYC@ZIF-90-FSH drug delivery system could target testis tissues, the LYC @ZIF-90 and the LYC@ZIF-90-FSH were labeled with Cy5.5, and then prepared with ethanol to obtain two solutions at a concentration of 4 mg/mL in advance. 8-week-old male Kunming mice were selected as subjects to inject 200 μL of the two solutions into the tail vein, and waited for preferably 4 h.

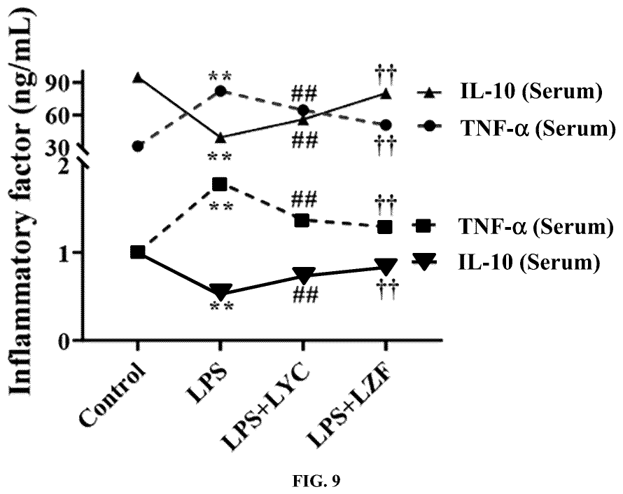

The mice after inhalation anesthesia were fixated and placed into an imaging camera obscura platform, the platform was raised and lowered to a suitable field of view by software; an illuminating lamp (bright field) was automatically turned on to take a first picture, followed by turning off the illuminating lamp to take a second picture in the dark (dark field), where the second picture had specific photons emitted by the mice. After superimposing background images of the bright field and the dark field, the location and intensity of the specific photons in the animal were visually displayed, as shown in FIG. 8. It was seen from FIG. 8 that under the same injection time, material concentration, and material amount, the FSH-loaded nanomaterials were more likely to enter the testis tissues of mice.

### Test Example 6 In Vivo Therapeutic Test of LYC@ZIF-90-FSH

A preparation method of the LYC @ZIF-90-FSH drug delivery system was the same as that in Example 3.

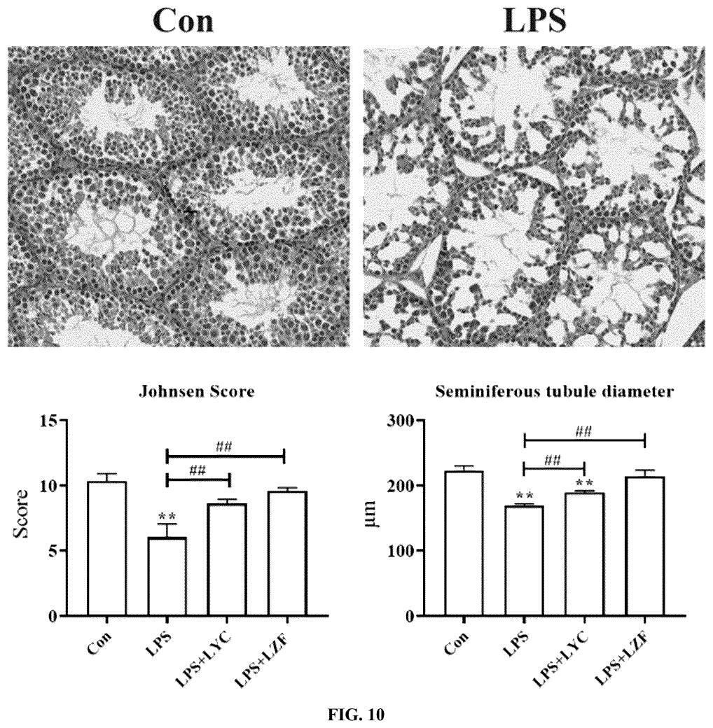

40 3-week-old male ICR mice were adaptively reared for one week, and randomly divided into 4 groups, including a control group (Con group), a 5 mg/kg LPS group (LPS group), a 5 mg/kg LYC+5 mg/kg/d LPS group (LYC+LPS group), and a 5 mg/kg LYC@ZIF-90-FSH+5 mg/kg/d LPS group (LZF+LPS group); one day after an acute challenge, the mice were sacrificed on a second day; for each mouse, blood was collected from eyeballs and a serum was preserved, and a testis tissue was rapidly stripped for subsequent detection. As shown in FIG. 9, results of an ELISA kit showed that an anti-inflammatory factor IL-10 in the serum and tissue increased after the intervention of LYC nanomaterials; and an anti-inflammatory factor TNF-α decreased significantly after treatment with the LYC nanomaterials.

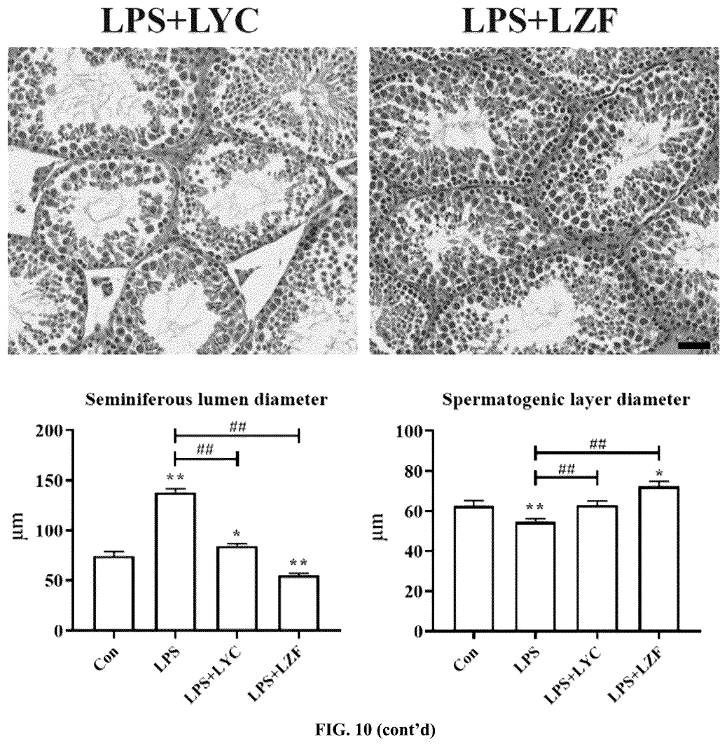

The testis tissue specimens were fixated with 10% formalin for 24 h, embedded in paraffin, cut into 5 μm thick glass slides, and stained with hematoxylin and eosin (HE) to observe histopathological changes. The results of HE staining were shown in FIG. 10. In the testis treated with LPS, the seminiferous tubules shrank and were disorderly arranged, and the spermatogenic cells fell off, which were significantly improved after LZF treatment.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A testis-targeted lycopene (LYC)/ZIF-90 nanocomposite, comprising an LYC/ZIF-90 nanocomposite and follicle-stimulating hormone (FSH) loaded on a surface and internal pores of the LYC-ZIF-90 nanocomposite; wherein
the LYC/ZIF-90 nanocomposite comprises a ZIF-90 metal-organic framework (MOF) material and LYC bound to the ZIF-90 MOF material through aldol condensation.

2. The testis-targeted LYC/ZIF-90 nanocomposite according to claim 1, wherein the ZIF-90 MOF material and the LYC have a mass ratio of 100:(10-15).

3. The testis-targeted LYC/ZIF-90 nanocomposite according to claim 1, wherein the ZIF-90 MOF material and the FSH have a mass ratio of 100:(5-10).

4. The testis-targeted LYC/ZIF-90 nanocomposite according to claim 2, wherein the ZIF-90 MOF material and the FSH have a mass ratio of 100:(5-10).

5. The testis-targeted LYC/ZIF-90 nanocomposite according to claim 1, wherein the ZIF-90 MOF material has a pore size of 1 nm to 3 nm.

6. The testis-targeted LYC/ZIF-90 nanocomposite according to claim 1, wherein the testis-targeted LYC/ZIF-90 nanocomposite has a particle size of 100 nm to 150 nm.

7. A preparation method of the testis-targeted LYC/ZIF-90 nanocomposite according to claim 1, comprising the following steps:
mixing the ZIF-90 MOF material and the LYC with an alcohol solvent to conduct aldol condensation to obtain an LYC/ZIF-90 nanocomposite; and
mixing the LYC/ZIF-90 nanocomposite and the FSH with the alcohol solvent to conduct loading to obtain the testis-targeted LYC/ZIF-90 nanocomposite.

8. The preparation method according to claim 7, wherein the ZIF-90 MOF material and the LYC have a mass ratio of 100:(10-15).

9. The preparation method according to claim 7, wherein the ZIF-90 MOF material and the FSH have a mass ratio of 100:(5-10).

10. The preparation method according to claim 8, wherein the ZIF-90 MOF material and the FSH have a mass ratio of 100:(5-10).

11. The preparation method according to claim 7, wherein the ZIF-90 MOF material has a pore size of 1 nm to 3 nm.

12. The preparation method according to claim 7, wherein the testis-targeted LYC/ZIF-90 nanocomposite has a particle size of 100 nm to 150 nm.

13. The preparation method according to claim 7, wherein the aldol condensation is conducted at 25° C. to 30° C. for 36 hours to 48 hours.

14. The preparation method according to claim 8, wherein the aldol condensation is conducted at 25° C. to 30° C. for 36 hours to 48 hours.

15. The preparation method according to claim 9, wherein the aldol condensation is conducted at 25° C. to 30° C. for 36 hours to 48 hours.

16. The preparation method according to claim 10, wherein the aldol condensation is conducted at 25° C. to 30° C. for 36 hours to 48 hours.

17. The preparation method according to claim 11, wherein the aldol condensation is conducted at 25° C. to 30° C. for 36 hours to 48 hours.

18. The preparation method according to claim 7, wherein a preparation method of the ZIF-90 MOF material comprises the following steps:
mixing an imidazole-2-carboxaldehyde solution, polyvinylpyrrolidone (PVP), and a zinc source solution to conduct complexation to obtain the ZIF-90 MOF material.

19. The preparation method according to claim 7, wherein the loading is conducted at 25° C. to 30° C. for 12 h to 24 h.

20. A method for treating male reproductive dysfunction, the method comprising applying the testis-targeted LYC/ZIF-90 nanocomposite according to claim 1 to a subject in need.
